(19) United States
(12) Patent Application Publication
(10) Pub. No.: US 2026/0316764 A1
(43) Pub. Date: Oct. 15, 2026

(54) METHOD AND SYSTEM FOR PREDICTING A STATE OF FATIGUE OF A DRIVER

(71) Applicant: Caterpillar Inc., Peoria, IL (US)

(72) Inventors: Tao MENG, Wuxi (CN); Lipeng JIANG, Wuxi (CN); Weixiu WANG, Wuxi (CN)

(21) Appl. No.: 19/485,869

(22) PCT Filed: May 1, 2024
(86) PCT No.: PCT/US2024/027122

§ 371 Date: Nov. 19, 2025

(30) Foreign Application Priority Data
May 26, 2023 (CN) 202310609430.2

(51) Int. Cl. B60W 40/08; B60W 50/00

(52) U.S. Cl. CPC B60W 40/08; B60W 50/0097; B60W 2040/0827; B60W 2040/0872; B60W 2050/0029; B60W 2300/17; B60W 2540/221; B60W 2540/223; B60W 2540/229

(57) ABSTRACT

The present invention relates to a method and a system for predicting a state of fatigue of a driver, in particular an operator of construction machinery, comprising the following steps: acquiring a data sequence of one fatigue parameter or data sequences of multiple fatigue parameters of the driver in at least one current time period; determining, based on the acquired data sequence(s), an optimal data of the one fatigue parameter or of the multiple fatigue parameters corresponding to the most fatigued state of the driver in each of the at least one current time period to generate a model input data in the time period; predicting, by using a trained time series prediction model for fatigue, a model prediction data of the one fatigue parameter or of the multiple fatigue parameters in an expected future time period after the at least one current time period based on the generated model input data; determining the state of the fatigue of the driver in the expected future time period based on the model prediction data. The system in particular for implementing the method of the present invention comprises a sensor device and a control device.

### TECHNICAL FIELD

The present invention relates to a method and a system for predicting a state of fatigue of a driver, in particular an operator of construction machinery.

### BACKGROUND

Various construction machinery, such as excavators, electric forklifts and loaders, are widely used in today's mining operations. Long sitting in a narrow and closed cab and doing tedious and repetitive work, operators of construction machinery are easy to get fatigued.

At present, most fatigue driving products in the market are used to monitor fatigue degrees of a motor vehicle driver in real time. The principle is that driver's facial features and behaviors are captured by cameras, and an alarm will be triggered when an improper driving behavior of the driver is identified.

However, this is more a response action that is forced to make when the driver has entered a fatigued state, rather than a proactive measure, i.e., an advance prediction of whether and when the driver will be tired. Therefore, the existing solution cannot completely eliminate the occurrence of accidents.

### SUMMARY OF THE INVENTION

Therefore, it is an object of the present invention to provide an improved method and system for predicting a state of fatigue of a driver, in particular an operator of construction machinery, by which the early warning function of driver's fatigue can be fulfilled.

The object is achieved by a method and a system with the following characteristics.

According to one aspect of the present invention, there is provided a method for predicting a state of fatigue of a driver, in particular an operator of construction machinery. The method comprises the following steps: acquiring a data sequence of one fatigue parameter or data sequences of multiple fatigue parameters of the driver in at least one current time period; determining, based on the acquired data sequence or data sequences, an optimal data of the one or the multiple fatigue parameters corresponding to the most fatigued state of the driver in each of the at least one current time period to generate a model input data in the time period; predicting, by using a trained time series prediction model for fatigue, a model prediction data of the one or the multiple fatigue parameters in an expected future time period after the at least one current time period based on the generated model input data; determining the state of the fatigue of the driver in the expected future time period based on the model prediction data.

According to another aspect of the present invention, there is provided a system for predicting a state of fatigue of a driver, in particular an operator of construction machinery, in particular a system designed for implementing the above-mentioned method, wherein the system comprises a sensor device and a control device. The sensor device is configured to acquire a data sequence of one fatigue parameter or data sequences of multiple fatigue parameters of the driver in at least one current time period; and the control device is configured to determine, based on the acquired data sequence or data sequences, an optimal data of the one or the multiple fatigue parameters corresponding to the most fatigued state of the driver in each of the at least one current time period to generate a model input data in the time period; predict, by using a trained time series prediction model for fatigue, a model prediction data of the one or the multiple fatigue parameters in an expected future time period after the at least one current time period based on the generated model input data; and determine the state of the fatigue of the driver in the expected future time period based on the model prediction data.

The method and system of the present invention can improve the prediction function of the existing anti-fatigue driving system. Thereby, drivers, in particular operators of construction machinery, can be well assisted in the working process, and can be reminded in time before fatigue driving or operation, so as to adjust their working states and avoid danger. In addition, through the method and system of the present invention, the fatigue of a driver in a certain future time can be predicted accurately in advance and the model calculation is more stable and reliable.

It will be appreciated that both the above general description and the following detailed description are exemplary and explanatory only and do not limit the scope of protection of the present invention.

### BRIEF DESCRIPTION OF THE DRAWINGS

The present invention is described in detail below with reference to the embodiments and in conjunction with the accompanying drawings.

FIG. 1 schematically shows an exemplary embodiment of a system for predicting the fatigue of an operator of a construction machine according to the present invention.

FIG. 2 schematically shows a flowchart of an exemplary embodiment of a method for predicting the fatigue of an operator of a construction machine according to the present invention.

### DETAILED DESCRIPTION OF THE EMBODIMENTS

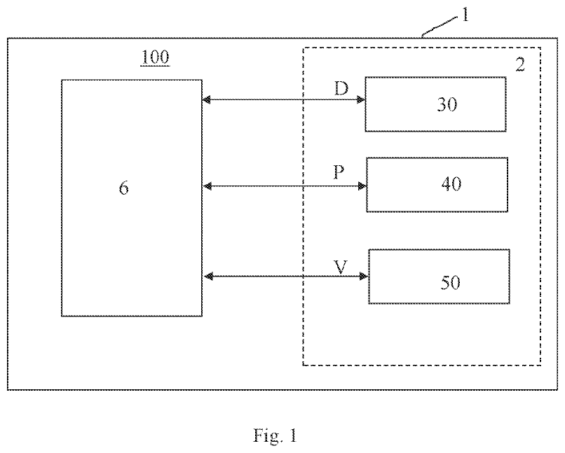

FIG. 1 schematically shows an exemplary embodiment of a system 100 for predicting a state of fatigue of an operator of a construction machine 1 (such as an excavator, an electric forklift, and a loader) in a future time based on a current state of the operator. The system 100 includes a sensor device 2 installed in the construction machine 1. The sensor device 2 is provided for detecting values or data of one or more fatigue parameters of the operator. The detection can be performed continuously or intermittently, for example, every few seconds or milliseconds. The one or more fatigue parameters reflect the state of the operator's fatigue, i.e., whether the operator is tired or not. For example, generally speaking, when an operator is gradually tired, his eye angle opening may become smaller, his head drooping angle will increase, and his eye closing time will become longer, which may be also accompanied by a decrease in the pressure of holding the operating lever, a decrease in the speed of the operating lever, and so on. For example, the fatigue parameter(s) may be selected from the group including biological parameters or operating parameters of an operator. The biological parameters can include, for example, the operator's eye angle opening, eye blinking frequency, head posture, pupil diameter, eyeball rotation speed and eye closing time. The operating parameters may include, for example, the operating lever pressure with which the operator holds the operating lever, the speed of the operating lever, and the like.

In this embodiment, three fatigue parameters are used, namely, the operator's eye angle opening, the operating lever pressure and the operating lever speed. These three fatigue parameters can most intuitively reflect the fatigue degree of the operator. For this reason, the sensor device 2 includes three sensors accordingly, namely, a camera 30, a pressure sensor 40 and a speed sensor 50. The camera 30 is configured to detect the eye angle opening data D when the operator works. The camera 30 can be installed in a cab of the construction machine 1 in a known manner, e.g. aligned with the operator's face. The pressure sensor 40 is provided to detect the pressure values P at which the operator holds the operating lever of the construction machine 1. The speed sensor 50 is provided to detect the speed values V at which the operator pushes the operating lever. The pressure sensor 40 and the speed sensor 50 may be installed on the operating lever.

The system 100 further comprises a control device 6. The sensor device 2 can communicate with the control device 6 to transmit, to the control device 6, the data D, P and V of the three fatigue parameters detected by the camera 30, the pressure sensor 40 and the speed sensor 50. The control device 6 is configured to generate a model input data (Ds, Ps, Vs) based on the received data D, P and V of the three fatigue parameters. The control device 6 is further configured to predict a model prediction data (Dd, Pp, Vv) of these three fatigue parameters at an expected future time using a trained time series prediction model for fatigue based on the generated model input data (Ds, Ps, Vs). The control device 6 is further configured to determine the state of the fatigue of the operator at the expected future time based on the model prediction data (Dd, Pp, Vv). The control device 6 can be arranged in the construction machine 1, for example, designed as a controller of the construction machine 1, or can be arranged outside the construction machine 1, for example, in a back end that communicates with the construction machine 1.

A time series prediction model, as its name implies, is an algorithmic model to predict an output data of a future time step based on one input data of a starting time step or a series of input data of a plurality of starting time steps. Therefore, the time series prediction model for fatigue can include two types. In a first type, the time series prediction model for fatigue calculates a model prediction data of an expected future time step from one model input data of a starting time step, wherein based on the model input data of the one or more fatigue parameters of the starting time step (a first time step), an output data of the one or more fatigue parameters of a next time step (a second time step) is calculated, and this intermediate output data of the second time step is used as a new input data to further calculate an output data of the one or more fatigue parameters of a third time step. This iteration continues until the model prediction data of the one or more fatigue parameters of the expected future time step is obtained.

Alternatively, in a second type, the time series prediction model for fatigue calculates a model prediction data in an expected future time step from multiple model input data of multiple starting time steps. Taking the case of three starting time steps as an example, an output data of a next time step (a fourth time step) is calculated from the three model input data of the three starting time steps (first, second, and third time steps), and then the model input data of the second and third time steps and the intermediate output data of the fourth time step are used as new input data to calculate an output data of a fifth time step. This iteration continues until the model prediction data of the expected future time step is obtained.

Here, a model input data contains the data of all fatigue parameters in a same time step. Therefore, in the case of multiple fatigue parameters, a model input data is actually a data group.

Each time step of the time series prediction model for fatigue is called a time period. Duration of a time period can be, for example, 1 second, 10 seconds, 30 seconds, 1 minute, 2 minutes, 3 minutes, 4 minutes or 5 minutes, and preferably 1 minute.

The time series prediction model for fatigue can include any suitable time series prediction model, typically a TCN (Temporal Convolutional Network) model or a LSTM (Long Short-term Memory Network) prediction model, etc.

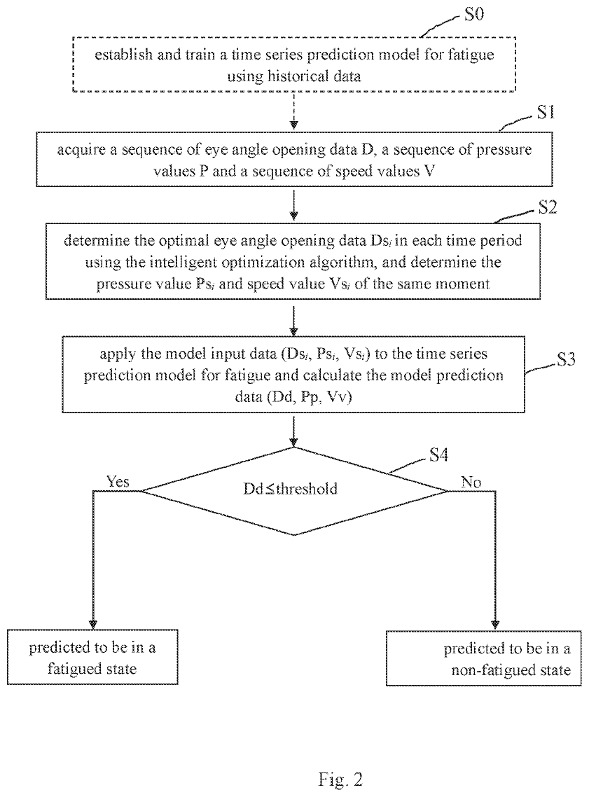

The method of the present invention is described in detail below with reference to the embodiment shown in FIG. 2.

FIG. 2 schematically shows a flowchart of an embodiment of the method according to the present invention for predicting a future state of fatigue of an operator by the system of FIG. 1. Before the method of the present invention starts, as a preparatory step SO, a time series prediction model for fatigue is first established and trained based on the historical data of one or more fatigue parameters. In this embodiment, three fatigue parameters are used, namely, the operator's eye angle opening detected by the camera 30, the operating lever pressure detected by the pressure sensor 40, and the operating lever speed detected by the speed sensor 50. Therefore, the historical data (D, P, V) of the operator's eye angle opening, the operating lever pressure, and the operating lever speed at a same moment are selected from their historical data to form a data group, and such multiple data groups form a times series to train the time series prediction model for fatigue.

After a trained time series prediction model for fatigue has been provided or existed, in a step SI, a data sequence for each of the three fatigue parameters (i.e. the operator's eye angle opening, the operating lever pressure, and the operating lever speed) is acquired in at least one current time period. That is, the current state of the operator's fatigue is acquired to predict how soon the operator will be tired from the current time on. It is well known that, as time goes by, the operator may gradually feel tired during the, e.g. 8-hour, work, but he may be also suddenly refreshed due to an unexpected event such as telephone or alarm clock ringing. Therefore, when the fatigue in a future time period is always predicted from the current state, a prediction result is more accurate.

Here, the at least one current time period should be understood as at least one time period from the current moment on, that is, one time period or multiple time periods. For example, if the duration of each time period is 1 minute, then the at least one current time period extends one minute or several minutes or even over ten minutes or dozens of minutes from the current moment on. The total number N (N=1, 2, 3, . . . ) of the at least one time period during which the three data sequences of the three fatigue parameters are acquired depends on the type of the time series prediction model, more precisely, it is equal to the number of the model input data, i.e., the number of data groups composed of data of the three fatigue parameters, of the time series prediction model. In other words, if the number of the model input data of the time series prediction model is four, i.e. a model prediction data in a future time period is predicted based on four model input data in four starting time periods (one model input data in each time period), then the three data sequences need to be acquired in four time periods from the current moment. If the time series prediction model has only one model input data, i.e. a model prediction data in a future time period is predicted based on only one model input data in one starting time period, then the three data sequences need to be acquired only in one time period from the current moment. The data sequence means that, for each fatigue parameter, its values are collected and determined at multiple moments within each time period to form a data sequence of the fatigue parameter.

In order to obtain these data sequences, the sensor device 2 such as the camera 30, the pressure sensor 40 and the speed sensor 50 can be used to detect in real time the time series of the eye angle opening data D, the operating lever pressure value P and the operating lever speed value V continuously or intermittently in at least one current time period. Here, the acquisition moments in the time series of the eye angle opening data D, the operating lever pressure value P and the operating lever speed value V may not be exactly the same.

Next, in step S2, based on the acquired data sequences of the three fatigue parameters, the control device 6 determines the optimal data of the three fatigue parameters corresponding to the most fatigued state of the operator in each of the at least one time period, so as to generate the model input data in respective time period(s). For each time period I (i=1, 2, . . . , N), here, the optimal solution Dsi of the first fatigue parameter, e.g. the eye angle opening, in this time period i can be calculated as a first optimal data using an intelligent optimization algorithm based on the data sequence of the eye angle opening data D acquired in this time period i. Here, the intelligent optimization algorithm can include any suitable local optimization algorithm, such as a whale algorithm, a particle swarm optimization algorithm, an artificial bee colony algorithm, an ant colony algorithm, an artificial fish swarm algorithm, a firefly algorithm and a gray wolf algorithm. Then, corresponding data of other fatigue parameters at the same time as the first optimal data (i.e. the optimal solution Dsi), such as the operating lever pressure data Psi and the operating lever speed data Vsi, are selected as other optimal data. Thus, a model input data (Dsi, Psi, Vsi) can be generated for each time period i, which is composed of the first optimal data Dsi and other optimal data Psi and Vsi in the time period i.

In step S3, the control device 6 predicts, based on the generated at least one model input data (Dsi, Psi, Vsi), where i=1, 2, . . . , N, a model prediction data (Dd, Pp, Vv) for an expected future time period (e.g. the minute one hour later from the current moment), using a trained time series prediction model for fatigue. The model prediction data includes the respective prediction data Dd, Pp, Vv of the three fatigue parameters. The expected future time period is later than the at least one current time period.

In step S4, the control device 6 determines the state of the operator's fatigue in the expected future time period based on the model prediction data (Dd, Pp, Vv). Here the state of the operator's fatigue in the expected future time period can be determined based on a comparison between the predicted data Dd of the first fatigue parameter, i.e. the eye angle opening, and a predetermined threshold. For example, when the predicted data Dd is less than or equal to the predetermined threshold, it can be considered that the operator will be in a fatigued state within the expected time period. Conversely, if the predicted data Dd is greater than the threshold, it can be considered that the operator will be in a non-fatigued state within the expected time period. Here the threshold is determined by the historical data of eye angle opening.

In another embodiment not shown, the state of the operator's fatigue in the expected future time period can be determined by a comparison between the predicted data Dd, Pp, Vv of all of the three fatigue parameters and the predetermined reference ranges of these fatigue parameters. For example, when the predicted data Dd, Pp and Vv of these three fatigue parameters all fall into their respective reference ranges [D1, D2], [P1, P2] and [V1, V2], that is, when DI≤Dd ≤D2, P1≤Pp <P2 and VI≤Vv ≤V2 are satisfied simultaneously, it can be considered that the operator will be in a fatigued state within the expected time period. Conversely, if at least one of Dd, Pp and Vv falls outside the reference ranges [D1, D2], [P1, P2] and [V1, V2], it can be considered that the operator will be in a non-fatigued state within the expected time period. Here, the predetermined reference ranges [D1, D2], [P1, P2] and [V1, V2] of the fatigue parameters are determined by the historical data of the respective fatigue parameters.

Three fatigue parameters are taken as examples here to illustrate the method and system of the present invention, but another number of fatigue parameters are also feasible. For example, the number of the fatigue parameters can be one, two, four or more. When there is only one fatigue parameter, e.g. the eye angle opening, this fatigue parameter is the first fatigue parameter. Therefore, when optimal data in a certain time period is to be determined, the optimal solution of the first fatigue parameter in the time period is calculated using the intelligent optimization algorithm based on the acquired data sequence of the first fatigue parameter, and is taken as the model input data in this time period. After the model prediction data in the expected future time period is predicted from the model input data, whether the operator is in a fatigued state is determined from a comparison between the first fatigue parameter and a predetermined threshold or a predetermined reference range. The first fatigue parameter is preferably selected from the biological parameters, such as eye angle opening, eye blinking frequency, head posture, pupil diameter, eyeball rotation speed and eye closing time.

The method and system of the present invention can also be used to predict the fatigue driving of a motor vehicle driver driving on a road. Here the fatigue parameters that can best reflect the state of the driver's fatigue are selected, including the driver's eye angle opening, eye blinking frequency, head posture, pupil diameter, eyeball rotation speed, eye closing time, the hands' grip force on the steering wheel and so on.

1. A method for predicting a state of fatigue of a driver, in particular an operator of construction machinery, characterized by comprising steps of:
acquiring a data sequence of one fatigue parameter or data sequences of multiple fatigue parameters of the driver in at least one current time period;
determining, based on the acquired data sequence(s), an optimal data of the one fatigue parameter or of the multiple fatigue parameters corresponding to the most fatigued state of the driver in each of the at least one current time period to generate a model input data in the time period;
predicting, by using a trained time series prediction model for fatigue, a model prediction data of the one fatigue parameter or of the multiple fatigue parameters in an expected future time period after the at least one current time period based on the generated model input data;
determining the state of the fatigue of the driver in the expected future time period based on the model prediction data.

2. The method according to claim 1, characterized in that the step of determining an optimal data of the one fatigue parameter or of the multiple fatigue parameters corresponding to the most fatigued state of the driver in each of the at least one current time period comprises:
calculating, based on the acquired data sequence of a first fatigue parameter of the one or the multiple fatigue parameters, an optimal solution of the first fatigue parameter in each time period as a first optimal data in the time period, using an optimization algorithm,
in particular, in each time period, selecting corresponding data of other fatigue parameter(s) of the multiple fatigue parameters at the same moment as the first optimal data, as other optimal data in the time period; and
using the first optimal data in each time period as the model input data in said time period, or combining the first optimal data and the other optimal data in each time period into a model input data in said time period.

3. The method according to claim 2, characterized in that the optimization algorithm comprises at least one of a whale algorithm, a particle swarm optimization algorithm, an artificial bee colony algorithm, an ant colony algorithm, an artificial fish swarm algorithm, a firefly algorithm and a grey wolf algorithm.

4. The method according to claim 1, characterized in that the step of determining the state of the fatigue of the driver in the expected future time period based on the model prediction data comprises:
determining, based on a comparison between the model prediction data of a first fatigue parameter of the one or the multiple fatigue parameters and a predetermined threshold, whether the driver is in a fatigued state in the expected future time period.

5. The method according to claim 4, characterized in that the predetermined threshold is determined by historical data of the first fatigue parameter.

6. The method according to claim 1, characterized in that the step of determining the state of the fatigue of the driver in the expected future time period based on the model prediction data comprises:
determining, based on a comparison between the model prediction data of the one or the multiple fatigue parameters and a predetermined reference range or predetermined reference ranges of the respective fatigue parameter(s), whether the driver is in a fatigued state in the expected future time period.

7. The method according to claim 6, characterized in that the predetermined reference range(s) of the respective fatigue parameter(s) is/are determined by historical data of the respective fatigue parameter(s).

8. The method according to claim 1, characterized in that the number of the at least one current time period is the same as the number of the model input data.

9. The method according to claim 1, characterized in that the step of acquiring a data sequence of one fatigue parameter or data sequences of multiple fatigue parameters in the at least one current time period comprises:
continuously or intermittently detecting sensor data of the one or the multiple fatigue parameters in the at least one current time period, in particular by a sensor device.

10. The method according to claim 9, characterized in that the sensor device comprises a camera, and/or a pressure sensor, and/or a speed sensor.

11. The method according to claim 1, characterized in that the time series prediction model for fatigue comprises a TCN model or an LSTM model.

12. The method according to claim 1, characterized in that the time series prediction model for fatigue is trained based on a time series of a data group consisting of historical data of the respective fatigue parameter(s) at the same moment or in the same time period.

13. The method according to claim 1, characterized in that the fatigue parameter(s) is/are selected from the group of biological parameters and operating parameters of the driver, in particular, the biological parameters include the driver's eye angle opening, eye blinking frequency, head posture, pupil diameter, eyeball rotation speed and eye closing time, and the operating parameters include an operating lever pressure with which the driver holds the operating lever, a grip force exerted by the driver on a steering wheel, and an operating lever speed.

14. The method according to claim 13, characterized in that three fatigue parameters are used, wherein a first fatigue parameter is selected from the biological parameters of the driver, in particular the driver's eye angle opening, and the other two fatigue parameters are the operating lever pressure and the operating lever speed.

15. A system for predicting a state of fatigue of a driver, in particular an operator of construction machinery, in particular a system for implementing the method according to claim 1, wherein the system comprises a sensor device and a control device,
characterized in that
the sensor device is configured to acquire a data sequence of one fatigue parameter or data sequences of multiple fatigue parameters of the driver in at least one current time period, and
the control device is configured to:
determine, based on the acquired data sequence(s), an optimal data of the one or the multiple fatigue parameters corresponding to the most fatigued state of the driver in each of the at least one current time period to generate a model input data in the time period;
predict, by using a trained time series prediction model for fatigue, a model prediction data of the one or the multiple fatigue parameters in an expected future time period after the at least one current time period based on the generated model input data; and
determine the state of the fatigue of the driver in the expected future time period based on the model prediction data.
